(19) United States
(12) Patent Application Publication
(10) Pub. No.: US 2003/0211603 A1
(43) Pub. Date: Nov. 13, 2003

(54) Reprogramming cells for enhanced differentiation capacity using pluripotent stem cells

(76) Inventors: David J. Earp, Oakland, CA (US); Melissa K. Carpenter, Castro Valley, CA (US); Joseph D. Gold, San Francisco, CA (US); Jane S. Lebkowski, Portola Valley, CA (US); J. Michael Schiff, Menlo Park, CA (US)

Correspondence Address: GERON CORPORATION, 230 CONSTITUTION DRIVE, MENLO PARK, CA, 94025

(21) Appl. No.: 10/344,680

(22) PCT Filed: Aug. 14, 2001
(86) PCT No.: PCT/US01/25493

(51) Int. Cl. C12N 5/08

(52) U.S. Cl. 435/366

(57) ABSTRACT

Described in this disclosure is a new process whereby cells of one tissue type can be reprogrammed to produce cells of a different tissue type. Cells from a human donor are reprogrammed by culturing adjacent to primate pluripotent stem cells (in an undifferentiated or newly differentiated state) or in an environment supplemented by components taken from pPS cells. Simultaneously or in a subsequent step, the donor cells can be treated in a manner that enhances differentiation towards a different tissue type. In this manner, patients in need of tissue regeneration can be treated with cells differentiated and reprogrammed from their own autologous cell donation.

### RELATED APPLICATIONS

This application is the 35 USC §371 National Stage of International Patent Application PCT/US01/25493, designating the U.S. and published as WO 02/14469 on Feb. 21, 2002. This application claims the priority benefit of U.S. provisional applications 60/225,509; 60/226,210; 60/225,511; 60/225,503; 60/225,622; and 60/225,510; all filed on Aug. 15, 2000.

The PCT application and the priority applications are hereby incorporated herein by reference in their entirety, as are U.S. patent application Ser. Nos. 09/859,291; 09/872,182; 09/872,183; and 60/252,688.

### TECHNICAL FIELD

This invention relates generally to the field of tissue regeneration and the biology of precursor cells. More specifically, it describes the reprogramming of cells to expand the range or alter the cell types that can be produced as progeny of the reprogrammed cells.

### BACKGROUND

Precursor cells have become a central interest in medical research. Many tissues in the body have a back-up reservoir of precursors that can replace cells that are worn out through normal function, or lost by injury or disease. Considerable effort has been made recently to isolate precursors of a number of different tissues for use in regenerative medicine.

U.S. Pat. No. 5,750,397 (Tsukamoto et al., Systemix) reports isolation and growth of human hematopoietic stem cells which are Thy-1+, CD34+, and capable of differentiation into lymphoid, erythroid, and myelomonocytic lineages.

U.S. Pat. No. 5,716,616 (Prockop et al.) proposes treating patients for a bone defect or the effects of bone marrow ablation by obtaining a bone marrow sample from a donor, isolating adherent cells of a stromal cell phenotype, and infusing them into the patient. U.S. Pat. No. 5,736,396 reports methods for lineage-directed differentiation of isolated human mesenchymal stem cells, using an appropriate bioactive factor. The derived cells can then be introduced into a host for mesenchymal tissue regeneration or repair.

U.S. Pat. No. 5,716,411 (Orgill et al.) relates to regenerating skin at the site of a burn or wound. The wound is first covered with a porous lattice such as a collagen glycosaminoglycan matrix, containing autologous or heterologous cells. Mesenchymal cells and blood vessels from healthy tissue under the burn site infiltrate the lattice. Then an epithelial autograft is placed over the site to form a neodermis and neoepidermis.

U.S. Pat. No. 5,766,948 (F. Gage) reports a method for producing neuroblasts from animal brain tissue. U.S. Pat. No. 5,672,499 (Anderson et al.) reports obtaining neural crest stem cells from embryonic tissue. U.S. Pat. No. 5,851,832 (Weiss et al., Neurospheres) reports isolation of putative neural stem cells from 8-12 week old human fetuses. U.S. Pat. No. 5,968,829 (M. Carpenter) reports human neural stem cells derived from primary central nervous system tissue.

U.S. Pat. No. 5,082,670 (F. Gage) reports a method for grafting genetically modified cells to treat defects, disease or damage of the central nervous system. Auerbach et al. (Eur. J. Neurosci. 12:1696, 2000) report that multipotential CNS cells implanted into animal brains form electrically active and functionally connected neurons. Brustle et al. (Science 285:754,1999) report that precursor cells derived from embryonic stem cells interact with host neurons and efficiently myelinate axons in the brain and spinal cord.

Recently, there have been hints that precursor cells of one lineage will form cells of another type when transplanted into a different tissue.

Bjornson et al. (Science 283:534, 1999; and U.S. Pat. No. 6,093,531) report that genetically labeled neural stem cells transplanted into irradiated hosts produced a variety of blood cells, including myeloid cells and lymphoid cells. Clarke et al. (Science 288:1660, 2000) report that labeled adult neurosphere cells injected into chick embryos appeared not only in the nervous system, but also in mesodermal derivatives such as mesonephros and heart muscle, and endodermal derivatives such as liver and intestinal epithelium.

Kulessa et al. (Genes Dev. 9:1250, 1995) investigated the transcription factor GATA-1, which is expressed in early hematopoietic progenitors but down-regulated in myelomonocytic cells. GATA-1 was reported to allow a v-myc transformed macrophage cell line to form myeloblasts, eosinophils, and erythroblasts. Geiger et al. (Cell 93:1055, 1998) reported that adult hematopoietic stem cells injected into mouse blastocysts acquired the ability to express fetal-type globin genes. Eglitis et al. (Proc. Natl. Acad. Sci. USA 94:4080, 1997) transplanted mice with bone marrow cells that were genetically marked. By in situ hybridization analysis, an influx of hematopoietic cells into the brain was detected. Some of the bone marrow derived cells in the tissue were found to stain for the microglial marker F4/80, or the astroglial marker GFAP.

Jackson et al. (Proc. Natl. Acad. Sci. USA 96:14482,1999) report hematopoietic potential of stem cells isolated from murine skeletal muscle. Gussoni et al. (Nature 401:390, 1999) report results of cell transplantation experiments in the mdx mouse model of Duchenne's muscular dystrophy. Injection of either normal hematopoietic stem cells or a population of muscle-derived cells into irradiated animals resulted in reconstitution of the hematopoietic compartment and partial restoration of dystrophin expression.

International Patent Publication WO 99/03973 (Pittenger et al., Osiris) proposes the use of mesenchymal stem cells to regenerate cardiomyocytes in vivo: The cells are genetically modified to enhance myocardial differentiation and integration. WO 99/19461 (Grompe) reports liver regeneration using pancreas cells. Aboseif et al. (Differentiation 65:113, 1999) cocultured human bladder urothelium with seminal vesicle mesenchyme of rat origin. Glandular structures were found in the human cells resembling prostate, and which were filled with secretions containing prostate-specific secretory proteins.

Alison et al. (Nature 406:257, 2000) reported analysis of liver tissue from female patients who had received a bone-marrow transplant from a male donor. Cells that were Y-chromosome positive were found in liver sections that also stained for the hepatocyte marker cytokeratin 8. The authors speculate that liver damage in the bone-marrow transplant patients promoted colonization and amplification of exogenous hematopoietic cells in the liver.

Theise et al. (Hepatology 32:11, 2000) analyzed archival autopsy and biopsy liver specimens from recipients of therapeutic bone narrow transplants and orthoscopic liver transplants. In situ hybridization analysis for chromosomal markers indicated that hepatocytes and cholangiocytes were derived from extrahepatic circulating cells, possibly of bone marrow origin. Petersen (Science 284:1168, 1999) followed hepatic regeneration in rats that had undergone bone marrow transplantation, and then subject to chemically induced hepatic injury. A proportion of the regenerated hepatic cells were found to be donor derived.

There is a need to develop new techniques for generating human tissue suitable for regenerative medicine for a variety of important clinical conditions. It would be advantageous to have a system to dependably obtain committed cells suitable for regenerative therapy from a readily available source of donor cells.

### SUMMARY OF THE INVENTION

This disclosure provides a system for reprogramming cells obtained from donor tissue so that they are no longer limited to the types of progeny to which they are otherwise committed. The reprogramming is effected by culturing a donor cell adjacent to primate pluripotent stem (pPS) cells (or cells newly differentiated from them). Alternatively or in addition, the medium may contain added components that are secreted from such cells, or can be recovered from the interior of such cells in the form of a lysate. The reprogrammed cells can simultaneously or in a subsequent procedure be differentiated into a new cell type suitable for readministration to the donor or another recipient for purposes of tissue regeneration.

One aspect of the invention is a method of reprogramming a human donor cell by culturing the cell in the presence of pPS cells, embryoid body cells, or a cell lysate or conditioned medium prepared from such cells. The donor cell can be a restricted lineage precursor cell, exemplified by various forms of CD34+ leukocytes, cord blood cells, mesenchymal stem cells, stromal cells, neural stem cells, and primary liver cells. In some circumstances, the donor cell or its progeny are cultured with or passaged into a growth environment that comprises an extracellular matrix and a medium supplemented with components that promote differentiation of the cell. The growth environment may also contain cells of a lineage different from the lineage of the cell being reprogrammed that assists differentiation of the donor cell towards a new phenotype, such as a neural cell or hepatocyte. Exemplary pPS cells are human embryonic stem (hES) cells, prepared as described later in this disclosure.

Another aspect of this invention is a reprogrammed human cell, prepared according to a method of this invention. Included is a human cell produced by culturing a cell ex vivo in the presence of a reprogramming component, specifically pPS cells, embryoid body cells, or a cell lysate or conditioned medium prepared from such cells. The reprogramming components can also be packaged separately and commercially distributed for the purpose of reprogramming donor cells.

Another aspect of the invention is a method for evaluating the effectiveness of a component (such as a medium, cell lysate, or cell population) in the reprogramming of a donor cell. The method comprises culturing the donor cell with the component, simultaneously or sequentially culturing the cell in a growth environment that promotes differentiation of the cell or its progeny into a new lineage, and then evaluating the effectiveness of the component based on whether the ability of the cell to produce progeny of the new lineage has been increased.

A further aspect of the invention is a method for screening a compound for toxicity or modulation potential for a particular cell type, in which the compound is combined with progeny of the particular cell type grown from a cell reprogrammed according to the invention, and then determining any phenotypic or metabolic changes in the progeny that result from contact with the compound.

Another aspect of the invention is a method for treating a patient to supplement activity of a particular cell type. The method involves obtaining patient cells of a different lineage from that of the cell type, reprogramming the cells according to this invention so that the reprogrammed cells can produce progeny of the particular cell type, and then readministering the reprogrammed cells to the patient.

Other aspects of the invention will be apparent from the description that follows.

### DRAWINGS

FIG. 1 is a line drawing depicting schemes for reprogramming human mesenchymal stem cells (hMSC) using human embryonic stem (hES) cells, and causing them to differentiate into cells bearing neuronal markers. In the Upper Panel, hMSC are mixed with undifferentiated hES cells, and then cultured together in conditions that allow mixed aggregates to form. They are then put through a two-step neuronal cell differentiation protocol, and analyzed for surface markers and cell genotype. In the Lower Panel, hMSC are cultured in a transwell apparatus opposite embryoid bodies (EB) obtained from hES cells. The two cell types are not in direct contact, but share the same culture medium.

FIG. 2 is a scanned image of a fluorescence micrograph, showing expression of the neuronal marker NCAM on hMSCs cocultured with hES-derived cells. The gray pattern shows NCAM expression. Detection of Y chromosome by FISH analysis produces the bright spots, and indicates which cells are derived from the hMSC in the coculture.

FIG. 3 is a line drawing depicting a scheme for reprogramming hMSC and causing them to differentiate into cells bearing hepatocyte markers. hMSC and hES cells are cultured so that mixed aggregates form. The dispersed cells are then differentiated according to a protocol using sodium n-butyrate (NaBut), which is known to cause pluripotent stem cells to produce hepatocytes.

FIG. 4 is a scanned image of a fluorescence micrograph, showing expression of albumin (a hepatocyte-specific marker) on hMSCs that have been reprogrammed by coculturing with hES derived cells, and put through the hepatocyte differentiation paradigm. After 17 days, cells were identified which were albumin positive and Y chromosome positive, showing they were derived from the hMSC cells in the population. In contrast, hMSC treated alone did not stain for albumin, showing that the ability to obtain hepatocytes from the hMSC depends on the step where the hMSCs are cocultured with the hES derived cells.

### DETAILED DESCRIPTION

A central challenge in using progenitor cells for tissue regeneration is tissue matching. Except in the case of autologous bone marrow transplantation, it is rare that a patient can be their own source of the cell type needed to regenerate the damaged tissue. Donations from any source other than a histoidentical sibling are mismatched to some degree, with potential for allograft rejection or graft-versus-host disease.

This invention provides a new process whereby cells of one tissue type can be reprogrammed to produce cells of a different tissue type. The cells are first obtained from a human donor or a tissue collection, and optionally stabilized in culture before the reprogramming. They are reprogrammed by culturing in an environment that includes pluripotent stem cells (either in an undifferentiated form, or as a heterogeneous populations of newly differentiated cells, such as embryoid bodies). Alternatively or in addition, the environment may contain conditioned medium or a cellular extract from such cells. Simultaneously or in a subsequent step, the cells being reprogrammed can be cultured in an environment that includes medium, substrate, soluble factors, or other cells that help direct the reprogrammed cell to differentiate towards a different tissue type. Cells reprogrammed according to this invention can be used in regenerative medicine, for drug screening, or for any other worthwhile purpose.

It is a hypothesis of this invention that factors present early in embryogenesis promote the capacity of embryonic cells to differentiate into all the progeny necessary to create a viable human being. When the nucleus of a suitably prepared adult cell is transferred into a suitable recipient cell, such as an oocyte, the combined cell can grow into a cloned adult that has the same genotype as the donor nucleus (WO 97/07669). Human embryonic stem cell lines prepared from human blastocysts have the capacity to grow in an undifferentiated form in culture almost indefinitely—yet preserve the ability to form tissues of all three germ layers (Thomson et al., Science 282:114,1998; U.S. Pat. No. 5,843,780). Human embryonic germ cells prepared from the genital ridge of an 8-11 week old fetus can also be maintained in long-term culture.

Without intending to be limited by theory, it is also hypothesized that factors present in the culture of such cells have the ability to reverse a cell's commitment towards a particular differentiation pathway—in effect, to begin a reprogramming process that restores to the cell an ability to make progeny with an expanded range of phenotypes. Simultaneously or subsequently, when the cell encounters growth conditions or agents that promote differentiation down another pathway, progeny of a new tissue type can be formed. These ideas are placed here for the interest of the reader, and do not need to be understood to put the invention into practice. The claimed invention is not restricted by mechanism of action, and is limited only by the features explicitly stated, or their equivalents.

This invention provides an elegant solution to the problem of histocompatibility in tissue regeneration. The process described in this disclosure enables human patients to become their own tissue donor. Cells are collected not from the diseased or damaged tissue, but from a different tissue that is relatively healthy and relatively easy to harvest. They are then reprogrammed (and further differentiated if necessary) to produce cells that are capable of repopulating the tissue where regeneration is needed. The cells are then administered back to the patient, potentially without needing to administer immunosuppressive therapy to prevent transplant rejection.

Definitions

“Reprogramming” is a process that confers on a cell a measurably increased capacity to form progeny of at least one new cell type, either in culture or in vivo, than it would have under the same conditions without reprogramming. This means that after sufficient proliferation, a measurable proportion of progeny having phenotypic characteristics of the new cell type if essentially no such progeny could form before reprogramming; otherwise, the proportion having characteristics of the new cell type is measurably more than before reprogramming. Under certain conditions, the proportion of progeny with characteristics of the new cell type may be at least ˜1%, 5%, 25% or more in the in order of increasing preference.

Before reprogramming, the cell can be a committed precursor cell capable of producing progeny of a certain lineage, or it can be a terminally differentiated cell, with or without proliferative capacity. After reprogramming, the cell can be a committed precursor cell capable of producing progeny of a different or expanded lineage, or it may be completely reprogrammed to a pluripotent cell. The reprogrammed cell may or may not have the capacity to make cells of the same lineage from which it was derived. As described below, reprogramming can be assessed by assessing the phenotype of the cell by characteristic morphological criteria, gene expression products, antigenic markers, or physiologic function—either directly, or after proliferation either in vivo or in an in vitro culture environment containing medium, matrix, and other factors typically used to culture cells of the desired type.

A primate pluripotent stem (pPS) cell is a cell that is capable of producing progeny that are derivatives of all of the three germinal layers: endoderm, mesoderm, and ectoderm. For certain uses described in this disclosure, pPS cells also have the capability of self-renewing in an undifferentiated state when cultured in vitro. Prototype “primate Pluripotent Stem cells” (pPS cells) are pluripotent cells derived from pre-embryonic, embryonic, or fetal tissue of the stated primate species at any time after gestation, which have the characteristic of being capable under the right conditions of producing progeny of several different cell types.

Non-limiting exemplars of pPS cells are rhesus and marmoset embryonic stem cells, as described by Thomson et al., Proc. Natl. Acad. Sci. USA 92:7844, 1995, human embryonic stem (hES) cells, as described by Thomson et al., Science 282:1145, 1998; and human embryonic germ (hEG) cells, described in Shamblott et al., Proc. Natl. Acad. Sci. USA 95:13726, 1998. Other types of pluripotent cells are included in the term that are pluripotent (that is, they are those capable of producing progeny that are derivatives of all of the three germinal layers), regardless of whether they were derived from embryonic tissue, fetal tissue, or adult tissue. In certain embodiments, pPS cells are limited to non-malignant cells, but otherwise it is permissible to use cells derived or differentiated from malignant tissue, tumors, or cells infected with oncovirus. Exemplary are human embryonal carcinoma (hEC) cells, described by Bronson et al., Cell Differ. 15:129, 1984, and reviewed by P. W. Andrews, J. Cell Biochem. 35:321, 1987. Both established cell lines and primary cultures are included in the term, providing they are fully pluripotent as defined earlier.

The term “embryoid bodies” (EBs) is a term of art synonymous with “aggregate bodies”. The terms refer to aggregates of differentiated and undifferentiated cells that appear when pPS cells overgrow in monolayer cultures, or are maintained in suspension cultures. Embryoid bodies are a mixture of different cell types, typically from several germ layers, distinguishable by morphological criteria. The term is understood to include a mixed cell population made from combining pPS cells with other cells (perhaps undergoing reprogramming), and allowing the mixed population to form aggregates in culture. Embryoid body (EB) cells are cells that are obtained from embryoid bodies, optionally disaggregated by mechanical or enzymatic means.

The terms “committed precursor cells”, “lineage restricted precursor cells” and “restricted developmental lineage cells” all refer to cells that are capable of proliferating and differentiating into several different cell types, but the range of their repertory is substantially more limited than pluripotent stem cells of embryonic origin capable of giving rise to progeny of all three germ layers. Examples of restricted lineage precursor cells are hematopoietic cells, which are pluripotent for blood cell types, and hepatocyte progenitors, which are pluripotent for sinusoidal endothelial cells, hepatocytes, and potentially other liver cells.

Cells are said to be “differentiated” when they do not have the capability of producing progeny of each of the three germinal layers. Instead, they are either committed precursor cells described above, or fully differentiated cells, which display the phenotype of a mature cell of a particular tissue. Examples of fully differentiated cells are neurons, hepatocytes, vascular endothelial cells, pancreatic islet cells, T and B lymphocytes, and keratinocytes. Fully differentiated cells may or may not retain the ability to proliferate.

A cell is described as “telomerized” if it has been manipulated to increase the level of telomerase reverse transcriptase (TERT) in a cell. For example, the cell can be genetically altered with a nucleic acid encoding a telomerase reverse transcriptase (TERT) of any species in such a manner that the TERT is transcribed and translated in the cell in a transient or continuous fashion, and combines with other components of the telomerase enzyme to elevate telomerase activity. Alternatively, telomerase activity may be increased by pulsing the cells with a TERT transcript or TERT protein. The term also applies to progeny of the originally altered cell that have an elevated level of TERT activity.

General Techniques

For further elaboration of general techniques useful in the practice of this invention, the practitioner can refer to standard textbooks and reviews in cell and tissue culture, embryology, and molecular biology.

General techniques in cell culture and media collection are outlined in Large Scale Mammalian Cell Culture (Hu et al., Curr. Opin. Biotechnol. 8:148, 1997); Serum-free Media (K. Kitano, Biotechnology 17:73,1991); Large Scale Mammalian Cell Culture (Curr. Opin. Biotechnol. 2:375,1991); and Suspension Culture of Mammalian Cells (Birch et al., Bioprocess Technol. 19:251, 1990). Textbooks on the subject include General Techniques in Cell Culture (Harrison & Rae, Cambridge, 1997); Animal Cell Culture Methods (Barnes & Mather, eds., Academic Press, 1998); Culture of Animal Cells (I. Freshney, 4th ed., John Wiley & Sons, 2000); Guidebook to the Extracellular Matrix, Anchor, and Adhesion Proteins (Kreis & Vale, eds., Oxford, 1999); Handbook of Cellular Manufacturing Systems (S. A. Irani, ed., John Wiley & Sons, 1999).

The properties, culture, and differentiation of embryonic stem cells are described in Teratocarcinomas and embryonic stem cells: A practical approach (E. J. Robertson, ed., IRL Press Ltd. 1987); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., al., 1993). Differentiation of stem cells is reviewed in Robertson, Meth. Cell Biol. 75:173, 1997; and Pedersen, Reprod. Fertil. Dev. 10:31, 1998. References that further describe the culturing of particular cell types are listed further on in the disclosure.

General biochemical techniques are described in Short Protocols in Molecular Biology (Ausubel et al., eds., 4th ed. 1999). Methods of protein chemistry are described generally in Protein Methods (Bollag et al., 1996); Guide to Protein Purification (Deutscher et al., eds., Methods Enzymol. vol. 182, Academic Press, 1997); Protein Analysis and Purification (I. M. Rosenberg, Springer Verlag, 1996).

Preparation and Growth of Pluripotent Stem Cells

Cultures of primate pluripotent stem (pPS) cells for use in this invention can be derived from pre-embryonic tissue (such as a blastocyst), embryonic tissue, or fetal tissue taken any time during gestation, typically but not necessarily before 10 weeks gestation.

Exemplary are human embryonic stem (hES) cells, which can be prepared as described by Thomson et al. (U.S. Pat. No. 5,843,780; Science 282:1145, 1998; Curr. Top. Dev. Biol. 38:133 ff., 1998; Proc. Natl. Acad. Sci. USA 92:7844, 1995). Human blastocysts can be obtained from human in vivo preimplantation embryos, in vitro fertilized (IVF) embryos, or one cell human embryos can be expanded to the blastocyst stage (Bongso et al., Hum Reprod 4: 706, 1989). The zona pellucida is removed from the blastocyst by brief exposure to pronase (Sigma). The inner cell masses are the isolated by immunosurgery, using a 1:50 dilution of rabbit anti-human spleen cell antiserum for 30 min, washing 3 times for 5 min in Dulbecco's modified Eagle's medium (DMEM), and then applying a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass by gentle pipetting, and the inner cell mass is plated on mouse embryonic fibroblasts.

ES cells can be maintained in 80% DMEM (Gibco #10829-018 or #11965-092), 20% defined fetal bovine serum (FBS) not heat inactivated, 1% non-essential amino acids, 1 mM L-glutamine, and 0.1 mM β-mercaptoethanol. Alternatively, ES cells can be maintained in serum-free medium, made with 80% Knock-Out DMEM (Gibco #10829-018), 20% serum replacement (Gibco #10828-028), 1% non-essential amino acids, 1 mM L-glutamine, and 0.1 mM β-mercaptoethanol. Just before use, human bFGF is added to a final concentration of ˜4 ng/mL (WO 99/20741).

Mouse embryonic fibroblasts (mEF) are obtained from CF1 mouse embryos at 13 days of pregnancy. Washed, dissected embryos are finely minced, and then digested for 5 min with trypsin. Feeder cells can be propagated in T150 flasks using mEF medium, containing 90% DMEM (Gibco #11965-092), 10% FBS (Hyclone #30071-03), and 2 mM glutamine. mEF are propagated in T150 flasks To prepare the feeder cell layer, plates are coated with 0.5% gelatin, plated near confluence, and rendered mitotically inactive (for example, with ˜4000 rads y-irradiation).

Nine to 15 days after isolation, outgrowths from the inner cell mass are dissociated into clumps either by exposure to calcium and magnesium-free PBS with 1 mM EDTA, by exposure to dispase or trypsin, or by mechanical dissociation with a micropipette; and then replated on mEF in fresh medium. Dissociated cells are replated on mEF feeder layers or extracellular matrix in fresh ES medium, and observed for colony formation. Colonies demonstrating undifferentiated morphology are individually selected by micropipette, mechanically dissociated into clumps, and replated. ES-like morphology is characterized as compact colonies that appear under the microscope as having a high nucleus to cytoplasm ratio and prominent nucleoli.

ES cells are routinely split every 1-2 weeks by brief trypsinization, exposure to Dulbecco's PBS (without calcium or magnesium and with 2 mM EDTA), exposure to Dispase or to Type IV Collagenase (˜200 U/mL; Gibco) or by selection of individual colonies by micropipette. Clump sizes of ˜50 to 2000 cells are optimal. Alternatively, after incubation with the protease, cultures can be scraped, dissociated into small clusters, and re-seeded onto fresh feeder cells at a split ratio of 1:3 to 1:30.

Other exemplary pPS cells are human Embryonic Germ (hEG) cells, from primordial germ cells present in human fetal material taken ˜8-11 weeks after the last menstrual period (Shamblott et al., Proc. Natl. Acad. Sci. USA 95:13726, 1998 and International Patent Publication WO 98/43679). Briefly, genital ridges are rinsed with isotonic buffer, then placed into 00.1 mL 0.05% trypsin/0.53 mM sodium EDTA solution (BRL) and cut into <1 mm3 chunks. The tissue is drawn through a 100 μL pipet tip to further disaggregate the cells, incubated at 37° C. for ˜5 min; then ˜3.5 mL growth medium is added. EG growth medium is DMEM, 4.5 mg/mL D-glucose, 2.2 mg/mL mM sodium bicarbonate; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/ml human recombinant basic fibroblast growth factor (bFGF, Genzyme); and 10 mM forskolin (in 10% DMSO).

Ninety-six well tissue culture plates are prepared with a sub-confluent layer of feeder cells cultured for 3 days in modified EG growth medium free of LIF, bFGF or forskolin, inactivated with ˜5000 rad g-irradiation. irradiation. Suitable feeders are STO cells (ATCC Accession No. CRL 1503). ˜0.2 mL of primary germ cell (PGC) suspension is added to each well. The first passage takes place after ˜7-10 days in EG growth medium, transferring each well to one well of a 24-well culture dish previously prepared with irradiated STO mouse fibroblasts. The cells are cultured with daily replacement of medium until cell morphology consistent with EG cells were observed, typically after 7-30 days or 1-4 passages.

ES and EG cells have characteristic antigens that can be identified by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., in Robertson E, ed. Teratocarcinomas and Embryonic Stem Cells. IRL Press, 207-246,1987). Pluripotency of embryonic stem cells can be confirmed by injecting approximately 0.5-10 10 6 cells into the rear leg muscles of 8-12 week old male SCID mice. Teratomas develop that demonstrate at least one cell type of each of the three germ layers.

Scientists at Geron Corporation have discovered that it is possible to grow pPS cells on an extracellular matrix with conditioned medium, as an alternative to growing them on feeder cells. The culture conditions promote proliferation of the pPS cells without differentiation. The medium can be prepared by culturing irradiated primary mouse embryonic fibroblasts at a density of ˜50,000 cm⁻² in medium containing 4 ng/mL bFGF. The culture supernatant is harvested after 1 day at 37° C., and supplemented with additional bFGF or other factors. Alternatives to primary fibroblasts for conditioning media include: 1) an embryonic fibroblast line that has been immortalized by transfecting with an expression cassette for telomerase reverse transcriptase; or 2) a human fibroblast-like cell line obtained by differentiating hES by forming embryoid bodies in suspension culture, then selecting and expanding cells with characteristics of fibroblasts.

In the absence of feeder cells, the pPS are plated onto a suitable substrate, such as one or more extracellular matrix components derived from basement membrane. A commercial preparation available from Becton Dickenson under the name Matrigel® is a soluble preparation from Engelbreth-Holm-Swarm tumor cells that gels at room temperature to form a reconstituted basement membrane. Also suitable is laminin, and other isolated extracellular matrix components. The pluripotent cells are typically plated at a density of ˜90,000 cm⁻² to 170,000 cm⁻² to promote survival and limit differentiation. During passage, enzymatic digestion is halted before cells become completely dispersed, and the cells are triturated until they are suspended as clumps of ˜10-200 cells. After passage, some cells around the periphery of colonies may differentiate, but cultures typically reestablish a larger proportion of undifferentiated cells. A typical population doubling time is ˜20-40 h.

Preparing Embryoid Bodies

General principles of culturing embryoid bodies are reported in O'Shea, Anat. Rec. (New Anat.) 257:323, 1999. pPS cells are cultured in a manner that permits aggregates to form, for which many options are available: for example, by overgrowth of a donor pPS cell culture.

Embryoid bodies are typically made in suspension culture, as follows. pPS cells are harvested by brief collagenase digestion, dissociated into clusters, and plated in non-adherent cell culture plates, in a medium composed of 80% KO DMEM (Gibco) and 20% non-heat-inactivated FBS (Hyclone), supplemented with 1% non-essential amino acids, 1 mM glutamine, 0.1 mM β-mercaptoethanol. Embryoid bodies are readily recognizable, being aggregates of heterogeneous differentiated cell population. The aggregates are fed every few days, and then harvested after a suitable period, typically 4-8 days. Optionally, the aggregates can then be transferred onto polyornithine-coated plates, and cultured for additional period to promote further differentiation and heterogeneity in the population, typically for 7 days.

Preparing Conditioned Medium

Medium can be conditioned for use according to this invention by culturing in it a population of undifferentiated pPS cells or embryoid body cells. The nature and time of the culture permits the cells to secrete into the medium at least one component that is effective in supporting the reprogramming of a suitable donor cell, as described below.

In optimal circumstances, the medium used for conditioning is selected to support the growth of both the pPS or embryoid body cells used for conditioning, and the cells subsequently to be reprogrammed. It should be recognized, however, that not all media support the cells being used to condition the medium without causing them to differentiate further. Accordingly, the medium is chosen in the first instance to support the pPS or embryoid body cells, and is adjusted if necessary after the conditioning and before it is used for reprogramming.

Typically, the cells used to condition the medium are first stabilized after the last passage by culturing for ˜2-3 days in standard medium until the culture is at least ˜50% confluent with the conditioning cells. If not present from the onset of the culture, the medium to be conditioned is then combined into the culture in an environment that allows the cells to release into the medium the components that support reprogramming. Optionally, the cells can be mitotically inactivated (i.e., rendered incapable of substantial replication) by radiation (e.g., ˜4,000 rads), treatment with a chemical inactivator like mitomycin c, or by any other effective method. This may facilitate keeping the cells at a higher density without further differentiation. However, inactivation of the cells is typically not necessary, since the medium is usually separated from the conditioning cells before use in reprogramming.

The cells are cultured in the medium for sufficient time to allow adequate concentration of released factors that effect reprogramming. Usually, medium is conditioned by culturing for 12, 24, 48, or 72 hours at 37° C. However, the culturing period can be adjusted upwards or downwards, determining empirically what constitutes an adequate period (for example, media conditioned for different periods can be tested for their ability to effect reprogramming, or for the concentration of essential factors). After collecting a batch of conditioned medium, the cells can sometimes be used to condition a further batch of medium over a further culture period, for as many cycles as desired as long as the cells retain their ability to condition the medium in an adequate fashion.

Selection of culture apparatus for conditioning medium can be made based on the scale and purpose of medium collection. In initial studies and for screening purposes, it is often convenient to produce cultured medium in standard culture flasks or multi-well plates. Large scale, automated, or GMP compliant production can involve the use of more specialized devices.

Continuous cell culture systems are reviewed by J. Furey (Genetic Eng. News 20:10, May 15, 2000). Perfusion culture involves removal of medium from the culture chamber, and replenishment with fresh medium. In the spin basket system, a basket-like device is attached to a drive shaft and covered by a porous screen through which medium can be exchanged. In the external filter perfusion system, a culture is circulated from a vessel, through a hollow-fiber filter module, and back to the vessel, with a pump attached to the loop to provide the circulation. A particular perfusion system, the ATF System (available commercially from Refine Technology, Edison N.J.) consists of a diaphragm pump on one end of a hollow-fiber housing, the other end of which is connected to a bioreactor. Alternating tangential flow through the fibers generates low shear laminar flow, which provides high flow rates, scalability, and adaptability to different bioreactors.

U.S. Pat. No. 4,501,815 reports a device for culturing differentiated cells. U.S. Pat. No. 4,296,205 reports cell culture and continuous dialysis flasks and their use. U.S. Pat. No. 5,994,129 reports a portable cassette for use in maintaining biological cells. U.S. Pat. No. 5,362,642 reports a containment system for storing, reconstituting, dispensing, and harvesting cell culture media. U.S. Pat. No. 6,022,742 reports a culture device and method. U.S. Pat. No. 6,058,721 reports a bioreactor for mammalian cell growth and maintenance, having a plurality of media outlets radially distributed throughout the bed, with media flow controlled by computer.

A particular embodiment of this invention is a device adapted for preparing conditioned medium, having a culture chamber containing cells of this invention capable of conditioning medium, and an outlet port for withdrawing medium from the culture chamber after conditioning by the cells. The device may also have a mass-transfer microporous surface in the form of a plate, a hollow fiber, or other structure that partitions the cultured cells from medium that has been conditioned, which allows free passage of the medium, and which provides passage to the outlet port. The device may also have one or more ports for introducing fresh medium, introducing additional cells, or removing expired cells and cell debris. For continuous flow systems, a pump may be attached to the medium inlet or outlet port to provide circulation.

Medium conditioned in this manner can be used for any worthwhile purpose, such as support and proliferation of stem cell populations of various kinds, and the reprogramming of cells as described below. If desired, the medium can be adapted to support the cells being treated or reprogrammed. This can include diluting in fresh medium, adding further nutrients, adding growth factors that support the cells before reprogramming, or adding growth factors or other components that promote differentiation of the cells along another pathway. In certain instances, it is also possible to condense high molecular weight solutes in the conditioned medium (for example, by microfiltration), or prepare an extract (for example, by salt precipitation, column chromatography, or lyophilization), and optionally stored or diluted in an appropriate buffer. The extract can then be added to new medium at a concentration sufficient for reprogramming.

Preparing Cell Lysate

Cell lysate or extract can be prepared from pPS cell cultures or embryoid body cells obtained as described earlier. For the sake of consistency, it is generally more convenient to use pPS cells grown without feeder cells. Before lysing, the cells typically are allowed to recover after the last passage by culturing for ˜2-3 days in standard medium until the culture is at least ˜50% confluent.

The cells can be lysed directly in the culture dish, for example, by replacing the medium with a solubilizing liquid, or by repeated freeze-thawing on a bath of dry ice. Alternatively, the cells can be resuspended from the culture surface before lysis, for example, by brief collagenase digestion as described earlier, or by scraping. The resuspended cells are collected, for example, by centrifugation, and then lysed by adding a suitable solvent, by freeze-thawing, by shearing through a narrow-gauge needle or in a tissue grinder, by sonicating, by mechanical homogenization, or by any other suitable method. Optionally, certain subcellular organelles can be removed or enriched, or membrane fractions can be prepared, according to standard methods. Techniques in subfractionating cells to produce cell components and extracts can be found in Storrie et al., Meth. Enzymol. 182:203, 1990; and in Subcellular Fractionation: A Practical Approach (Grahan & Rickwood, eds., Oxford, 1997).

The cell extract is then prepared for combining with the cells to be reprogrammed. Viscosity caused by long-chain nucleic acids can be reduced by treating with DNAse, or other appropriate nucleases. Non-ionic detergents with a high critical micelle concentration (such as sodium deoxycholate) can be removed by dialysis. Other detergents (such as Triton™ X-100, octyl glucoside, or Nonidet™-P40) can be removed, for example, on adsorbent beads or chromatography columns (Trescec et al., J. Chromatogr. A 852:87, 1999; K. Ohlendieck, Meth. Mol. Biol. 59:305, 1996; Guerrier et al., J. Chromatog. B Biomed. Appl. 664:119, 1995). Large particulates can be removed, for example, by centrifugation or microfiltration. As another option, high molecular weight solutes can be concentrated from the clarified extract (for example, by microfiltration, salt precipitation, column chromatography, or lyophilization).

The prepared lysate is then added at an effective concentration to a suitable medium that contains nutrients and other factors necessary to support culturing of the cells being reprogrammed.

Cells Suitable for Reprogramming

The technology described in this application can be employed for reprogramming a range of different cell types. In particular embodiments, progenitor cells of one lineage are reprogrammed to expand the range of progeny within the lineage, or to permit them to form progeny of a different lineage. Various types of lineage restricted precursor cells may be amenable to reprogramming under appropriate conditions. A number of types of lineage restricted precursor cells are known, and methods to obtain them are published. Cells suitable for reprogramming may include but are not limited to the particular cell types exemplified below.

U.S. Pat. No. 5,851,832 reports multipotent neural stem cells obtained from brain tissue. U.S. Pat. No. 5,766,948 reports producing neuroblasts from newborn cerebral hemispheres. U.S. Pat. Nos. 5,654,183 and 5,849,553 report the use of mammalian neural crest stem cells. U.S. Pat. No. 6,040,180 reports in vitro generation of differentiated neurons from cultures of mammalian multipotential CNS stem cells. WO 98/50526 and WO 99/01159 report generation and isolation of neuroepithelial stem cells, oligodendrocyte-astrocyte precursors, and lineage-restricted neuronal precursors. U.S. Pat. No. 5,968,829 reports neural stem cells obtained from embryonic forebrain and cultured with a medium comprising glucose, transferrin, insulin, selenium, progesterone, and several other growth factors.

Primary liver cell cultures can be obtained from human biopsy or surgically excised tissue by perfusion with an appropriate combination of collagenase and hyaluronidase. Alternatively, EP 0 953 633 A1 reports isolating liver cells by preparing minced human liver tissue, resuspending concentrated tissue cells in a growth medium and expanding the cells in culture. The growth medium comprises glucose, insulin, transferrin, T3, FCS, and various tissue extracts that allow the hepatocytes to grow without malignant transformation. The cells in the liver are thought to contain specialized cells including liver parenchymal cells, Kupffer cells, sinusoidal endothelium, and bile duct epithelium, and also precursor cells (referred to as “hepatoblasts” or “oval cells”) that have the capacity to differentiate into both mature hepatocytes or biliary epithelial cells (L. E. Rogler, Am. J. Pathol. 150:591, 1997; M. Alison, Current Opin. Cell Biol. 10:710,1998; Lazaro et al., Cancer Res. 58:514,1998).

U.S. Pat. No. 5,192,553 reports methods for isolating human neonatal or fetal hematopoietic stem or progenitor cells. U.S. Pat. No. 5,716,827 reports human hematopoietic cells that are Thy-1 positive progenitors, and appropriate growth media to regenerate them in vitro. U.S. Pat. No. 5,635,387 reports a method and device for culturing human hematopoietic cells and their precursors. U.S. Pat. No. 6,015,554 describes a method of reconstituting human lymphoid and dendritic cells.

U.S. Pat. No. 5,486,359 reports homogeneous populations of human mesenchymal stem cells that can differentiate into cells of more than one connective tissue type, such as bone, cartilage, tendon, ligament, and dermis. They are obtained from bone marrow or periosteum. Also reported are culture conditions used to expand mesenchymal stem cells. WO 99/01145 reports human mesenchymal stem cells isolated from peripheral blood of individuals treated with growth factors such as G-CSF or GM-CSF.

Other progenitor cells that may be suitable include but are not limited to chondrocytes, osteoblasts, and keratinocytes.

This invention further contemplates reprogramming of fully differentiated cells, such as fibroblasts from various tissues, smooth and skeletal muscle cells, monocytes and macrophages from various tissues, and endothelial cells. Also contemplated is reprogramming of cell populations that contain both fully differentiated cells and progenitor cells. Mixed cell populations include primary liver cell preparations, skin tissue, and samples from the mucosal epithelium. To be suitable for reprogramming, the cell of interest will have the capacity to proliferate, or will be rendered capable of proliferation, for example, by telomerization. Testing of various cell types and protocols for the purpose of reprogramming is described below.

Reprogramming Cells by Coculturing with pPS Cells or Embryoid Body Cells

The process of reprogramming is initiated by culturing the cell being reprogrammed in juxtaposition with pluripotent stem cells, or with embryoid body cells, prepared as described above.

The culture environment will contain a substrate and a nutrient medium that supports both the human donor cell being reprogrammed, and the pPS or embryoid body cells being used to cause the reprogramming. Where undifferentiated pPS cells are used, it is often preferable to have an environment in which pPS cells maintain their undifferentiated phenotype without feeder cells being present, since this avoids complications and facilitates later processing. In one illustration, hES cells are passaged on a matrix of Matrigel® or laminin, and supported by replacement of the medium every 24 h with medium conditioned by an immortalized mouse embryonic fibroblast line. After the last passage, the hES cells are cultured long enough for them to recover (˜2 days) but not to the extent that the cultures exceed confluence and the hES cells begin to differentiate.

The cells to be reprogrammed are suspended in ES medium, and layered onto the established hES cell culture. Once they have had a chance to adhere, the culture is fed with fresh conditioned medium, and culturing continues. In instances where the cells are non-adherent, they can be kept in suspension above the hES cells, or separated by a porous partition. It is also possible to reverse the process of combining the cultures. The hES cells are harvested as described earlier and triturated into small clumps of cells, and then added to the cells to be reprogrammed, already established in an environment that supports hES cell growth without differentiation.

Where embryoid body (EB) cells are used to cause the reprogramming, the culture environment will contain a substrate and a nutrient medium that simultaneously supports both EB cells and the cells being reprogrammed. In one illustration, EBs are first prepared from hES cells in suspension culture. The cells to be reprogrammed are suspended in medium, which is mixed with a suspension of EBs. The suspension is then plated onto a matrix that supports both cell types. Differentiated cells then grow out of the EB, and mingle with the other cells. In a variation of this, the EBs are dispersed into a single cell suspension before mixing, for example, by mild enzymatic digestion and trituration. In another illustration, a suspension of one of the cell populations is layered on to an established culture of the other cell population before it reaches confluence.

The human donor cell being reprogrammed, and the undifferentiated pPS or EB cells are cocultured for sufficient time and at a sufficient ratio to effect the reprogramming. It is believed that pPS or EB cells will reprogram about an equal number of other cells, although ratios of 1:10, 10:1, and so on are also contemplated. It is believed that a typical culture time to reprogram committed precursor cells will be ˜3 days—although periods of 1 day, 2 days, 5 days, or 1 or more weeks are contemplated, depending on several factors, such as the degree to which cells must be reprogrammed from commitment to the previous lineage. The optimal cell ratio and minimum coculturing time can be determined empirically, as a matter of routine optimization. For long culture periods, it will be necessary to replace the conditioned medium at regular intervals (typically 24 hour periods), and to passage the cells as necessary depending on the rate of proliferation of both the cell types. In some instances, it may be appropriate to mitotically inactivate the reprogramming cells (for example, with ˜4,000 rads y-irradiation, or treatment with mitomycin c to prevent outgrowth of the cells being reprogrammed.

In many instances, it is desirable not only to lessen commitment of the cells to their original tissue type, but also to promote differentiation towards a new tissue type. In some instances, this may occur at the same time as coculturing with the reprogramming pPS or EB cells; in other instances, it occurs at a subsequent time. Depending on the cell type desired, differentiation towards the new tissue type may involve growing the cells on an extracellular matrix or with medium components that promote differentiation along the developmental pathway of the new lineage. Many of these factors will also promote further differentiation of the reprogramming cells, which may or may not be a problem. As an alternative, the pPS cells can be rendered mitotically inactive, or the process may be conducted in two stages: first, coculturing of the cells to be reprogrammed with the reprogramming cells in an environment that limits differentiation of the reprogramming cells; followed by a second culturing stage (potentially after outgrowth or removal of the reprogramming cells), where the culture conditions promote differentiation of the cells being reprogrammed down the new lineage.

Adjunct techniques useful in the derivation of particular cell types can be inferred by analogy from papers describing certain types of precursor cells. General principals for obtaining tissue cells from pluripotent stem cells are reviewed in Pedersen, Reprod. Fertil. Dev. 6:543, 1994, and in WO 98/43679. For neural progenitors, neural restrictive cells and glial cell precursors, see Bain et al., Biochem. Biophys. Res. Commun. 200:1252, 1994; Trojanowski et al., Exp. Neurol. 144:92, 1997; Wojcik et al., Proc. Natl. Acad. Sci. USA 90:1305-130; and U.S. Pat. Nos. 5,851,832, 5,928,947, 5,766,948, and 5,849,553. For cardiac muscle and cardiomyocytes see Chen et al., Dev. Dynamics 197:217, 1993 and Wobus et al., Differentiation 48:173,1991. For hematopoietic progenitors, see Burkert et al., New Biol. 3:698, 1991 and Biesecker et al., Exp. Hematol. 21:774, 1993. U.S. Pat. No. 5,773,255 relates to glucose-responsive insulin secreting pancreatic beta cell lines. U.S. Pat. No. 5,789,246 relates to hepatocyte precursor cells. Other progenitors of interest include but are not limited to chondrocytes, osteoblasts, retinal pigment epithelial cells, fibroblasts, skin cells such as keratinocytes, dendritic cells, hair follicle cells, renal duct epithelial cells, smooth and skeletal muscle cells, testicular progenitors, and vascular endothelial cells.

Scientists at Geron Corporation have discovered that culturing pluripotent stem cells in the presence of ligands that bind growth factor receptors promotes enrichment for neural precursor cells. The growth environment may contain a neural cell supportive extracellular matrix, such as fibronectin. Suitable growth factors include but are not limited to EGF, bFGF, PDGF, IGF-1, and antibodies to receptors for these ligands. The cultured cells may then be optionally separated based on whether they express a marker such as A2B5. Under the appropriate circumstances, populations of cells enriched for expression of the A2B5 marker have the capacity to generate both neuronal cells (including mature neurons), and glial cells. Optionally, the cell populations are further differentiated, for example, by culturing in a medium containing an activator of cAMP.

Scientists at Geron Corporation have also discovered that culturing pluripotent stem cells in the presence of a hepatocyte differentiation agent promotes enrichment for hepatocyte-like cells. The growth environment may contain a hepatocyte supportive extracellular matrix, such as collagen or Matrigel® matrix. Suitable differentiation agents include various isomers of butyrate and their analogs, exemplified by n-butyrate. The cultured cells are optionally cultured simultaneously or sequentially with a hepatocyte maturation factor, such as an organic solvent like dimethyl sulfoxide (DMSO); a maturation cofactor such as retinoic acid; or a cytokine or hormone such as a glucocorticoid, epidermal growth factor (EGF), insulin, TGF-α, TGF-β, fibroblast growth factor (FGF), heparin, hepatocyte growth factor (HGF), IL-1, IL-6, IGF-I, IGF-II, and HBGF-1.

In addition or as an alternative, differentiation of the cells towards a new tissue type can be promoted by placing them with instructor cells supportive for the new tissue type. In some instances, the instructor cells will be the same type as the cell being reprogrammed. For example, it may be helpful to culture cells being reprogrammed into neural progenitors with other types of neural cells. In other instances, it will be a feeder or matrix-forming cell typical of the environment of the new cell type in vivo. For example, hematopoietic stem cell differentiation can be regulated by the use of mesenchymal stem cells (WO 99/64565, WO 99/61588). After the process is complete, instructor cells can be removed by a suitable technique, such as complement-mediated lysis, affinity or adherence techniques, antibody or ligand-receptor labeling followed by fluorescence-activated cell sorting, centrifugation through differential gradients, or selection with drugs to which the instructor cells have been rendered sensitive. The role of the instructor cell may also be fulfilled after administration of the reprogrammed cells into a particular environment in vivo.

For most purposes, before the reprogrammed cells are put into use, it will be desirable to remove the pPS or EB cells used for reprogramming. The cell population is described as “substantially pure” if less than ˜5% of the population has a genotype that is different from that of the reprogrammed cells. Preferably, the population will have <1% or <0.2% cells of a different genotype, in order of increasing preference.

Under certain circumstances, the reprogrammed cells may become substantially pure just by outgrowing the reprogramming cells and their progeny. In other circumstances, it will be necessary to invoke a separation procedure. If the cells differ sufficiently in morphological characteristics, it may be possible to separate them by physicochemical means: for example, separation on a density gradient such as Ficoll®, or adherence to a surface. Specific methods of eliminating the pPS or EB cells, or positively selecting the reprogrammed cells, include antibody recognition of expressed markers, or ligand interaction with cell surface receptors. Specific binding can then serve as the basis of affinity separation, agglutination, complement-mediated lysis, or fluorescence-activated separation of one of the two cell types. Markers characteristic of undifferentiated pPS cells include SSEA-1 (for human embryonic germ cells), SSEA-3, SSEA-4, Tra-1-60, Tra-1-81, Oct-4 (for hES cells), or telomerase reverse transcriptase. Markers characteristic of reprogrammed cells relate to the new tissue type, and are illustrated in the next section.

It is worthwhile to keep in mind that undifferentiated pPS cells are pluripotent, while EB cells represent a heterogeneous mixture of a variety of cell types. Accordingly, some of these cells may differentiate down the same lineage as the cells being reprogrammed. When this occurs, some of the phenotypic markers on the reprogrammed cells may be shared on differentiated pPS or EB cells. To neutralize the effect of parallel differentiation, a marker can be selected that depends on genotypic differences between the two cells. Such markers include blood group antigens, histocompatibility alloantigens, and allotypic variants on other surface proteins. Also useful are antigens that are species-specific (if the pPS cells are derived from a different species as the cells being reprogrammed), and X and Y chromosomal markers (if the pPS cells are of a different sexual genotype).

As an alternative, it is possible to eliminate pPS cells by drug selection if the pPS cells are rendered susceptible to the drug before use in reprogramming. Suitable methods for transferring vector plasmids into hES cells include lipid/DNA complexes, such as those described in U.S. Pat. Nos. 5,578,475; 5,627,175; 5,705,308; 5,744,335; 5,976,567; 6,020,202; and 6,051,429. Exemplary is the formulation Lipofectamine 2000™, available from Gibco/Life Technologies Cat. #11668019. Another exemplary reagent is FuGENE™ 6 Transfection Reagent, a blend of lipids in non-liposomal form and other compounds in 80% ethanol, obtainable from Roche Diagnostics Corporation (cat #1 814 443). The plasmid will contain a drug susceptibility gene, such as Herpes virus thymidine kinase, and pPS cells and their progeny can subsequently be from mixed cell populations by treating with ganciclovir. Conversely, for testing purposes or for in vitro use, the cells being reprogrammed can themselves be genetically altered with a resistance gene to a drug such as hygromycin, neomycin, or puromycin, and the corresponding drug can then be used to eliminate the pPS cells.

Another embodiment of the invention is a kit for reprogramming a human cell, comprising pPS or embryoid body cells in a suitable container, optionally accompanied by or distributed in conjunction with written indications for use of the medium for reprogramming. The indication may be in any form that indicates that reprogramming is one of the possible uses of the product, such as a label, a product insert, or accompanying instructions or advertising.

Using Conditioned Medium to Reprogram Cells

In some instances, cells can be reprogrammed by culturing them in an environment that includes conditioned medium from pPS or embryoid body cells, prepared as described earlier. The conditioned medium can be supplemented before use with additional nutrients or growth factors beneficial to the cells being reprogrammed. Possible inclusions are pan-specific growth factors such as basic fibroblast growth factor (bFGF) or epidermal growth factor (EGF). Other possible inclusions are growth factors or other components that help support cells of the type being reprogrammed. During the reprogramming process, the cells may become less dependent on tissue-specific factors for the tissue from which they were derived, and more dependent on factors that support the new cell type.

The cells are cultured in the conditioned medium for sufficient time to effect the reprogramming. It is believed that a culture time of at least 24 hours will generally be necessary. Longer culture times of 2, 5 or 20 days, or even longer, are contemplated, depending on several factors, such as the degree to which cells must be reprogrammed from commitment to the previous lineage. The minimum culturing time can be determined empirically, as a matter of routine optimization. For long culture periods, it will be necessary to replace the conditioned medium at regular intervals (typically 24 hour periods), and to passage the cells as necessary depending on the rate of proliferation.

Simultaneous or subsequent differentiation of the reprogrammed cells into a new cell lineage can occur as described in the previous section.

This invention also contemplates protocols using a succession of different cultured media. In one embodiment, the cells to be reprogrammed are cultured in conditioned medium that helps reduce commitment to the previous cell lineage, and then in a second medium that continues the reprogramming and/or promotes differentiation towards the new cell type. The new medium may be from a precursor cell mixture (such as embryoid body cells), or from a mature cell line (such as the HepG2 hepatocyte line) that provides factors found in the natural environment of the new cell type and takes the place of an instructor cell. This invention also contemplates protocols in which conditioned medium is taken not from pPS cells or embryoid bodies, but from committed precursor cells (differentiated from pPS cells or from another source), which simultaneously reduces commitment of the cells being reprogrammed from the old lineage, and promotes commitment to the new lineage.

Another embodiment of the invention is a kit for reprogramming a human cell, comprising conditioned medium in a suitable container, optionally accompanied by or distributed in conjunction with written indications for use of the medium for reprogramming. The indication may be in any form that indicates that reprogramming is one of the possible uses of the product, such as a label, a product insert, or accompanying instructions or advertising.

Using Cell Lysate to Reprogram Cells

In some instances, cells can be reprogrammed by culturing them in an environment that includes a basal medium containing soluble lysate prepared from hES cells or embryoid body cells. Solubilized lysate is added to a medium containing nutrients, growth factors, and other supplements appropriate for the cell being reprogrammed. Alternatively, a growth environment is prepared comprising the lysate, and the cells are passaged into the new environment. It may also be possible to incorporate components of a cell extract into the matrix that supports the cells.

The cells are cultured with an amount of lysate prepared from pPS cells or embryoid bodies for a sufficient time to effect reprogramming. It is believed that lysate prepared from a certain number of cells will be adequate to reprogram an equal number of partially differentiated cells. More concentrated or more dilute solutions may be appropriate, depending on such factors as the concentration of the lysate, the extent to which the lysate has been processed, and the geometry of the culture environment. It is believed that a culture time of at least 24 hours will generally be necessary. Longer culture times of 2, 3, or 5 days, or even longer, are contemplated, depending on several factors, such as the degree to which cells must be reprogrammed from commitment to the previous lineage. The amount of lysate and the culturing time needed to effect reprogramming can be determined empirically, as a matter of routine optimization. For long culture periods, it will be necessary to replace the lysate-containing medium at regular intervals (typically 24 hour periods), and to passage the cells as necessary depending on the rate of proliferation.

Simultaneous or subsequent differentiation of the reprogrammed cells into a new lineage can occur as described earlier.

This invention also contemplates protocols using a succession of different cell lysates or extracts. In one embodiment, the cells to be reprogrammed are cultured in cell lysate that helps reduce commitment to the previous cell lineage, and then in a second medium that continues the reprogramming and/or promotes differentiation towards the new cell type. The new extract may be from a precursor cell mixture, or from a mature cell line (such as the HepG2 hepatocyte line) that provides factors found in the natural environment of the new cell type and takes the place of an instructor cell. This invention also contemplates protocols in which a cell extract is taken not from undifferentiated pPS cells or embryoid body cells, but from committed precursor cells (differentiated from pPS cells or from another source), which simultaneously reduces commitment of the cells being reprogrammed from the old lineage, and promotes commitment to the new lineage.

Another embodiment of the invention is a kit for reprogramming a human cell, comprising cell lysate in a suitable container (optionally diluted in a suitable buffer or other excipient, or provided in solid form). The container will typically be accompanied by or distributed in conjunction with written indications for use of the medium for reprogramming. The indication may be in any form that indicates that reprogramming is one of the possible uses of the product, such as a label, a product insert, or accompanying instructions or advertising.

Assay Methods for Effective Reprogramming

Developing protocols for reprogramming of cells according to this invention may involve optimization of culture conditions as a matter of routine experimentation. This type of developmental work is often facilitated by setting up an assay that provides a relatively rapid readout whenever reprogramming is successful.

An assay method suitable for evaluating the effectiveness of reprogramming typically involves conducting a reprogramming process with a set of reprogramming reagents, and then evaluating the effectiveness of the process or the reagents on whether the ability of the cell to produce progeny of the new lineage has been increased. Progeny of a new lineage can be detected by the presence of a phenotypic marker characteristic of the new line but absent (or less prominent) on the cell and its progeny before reprogramming.

Markers that occur on various types of undifferentiated pluripotent cells include SSEA-1, SSEA-3, SSEA-4,Tra-1-60, Tra-1-81, alkaline phosphatase activity, Oct-4, and telomerase reverse transcriptase.

Committed precursors and differentiated cells can be recognized by characteristic morphology and the markers they express. For skeletal muscle: myoD, myogenin, and myf-5. For endothelial cells: PECAM (platelet endothelial cell adhesion molecule), Flk-1, tie-1, tie-2, vascular endothelial (VE) cadherin, MECA-32, and MEC-14.7. For smooth muscle cells: specific myosin heavy chain. For cardiomyocytes: GATA-4, Nkx2.5, cardiac troponin I, a-myosin heavy chain, and ANF. For pancreatic cells, pdx and insulin secretion. For hematopoietic cells and their progenitors: GATA-1, CD34, β-major globulin, and β-major globulin like gene bH1.

Markers for neural cells include β-tubulin III, neurofilament, or microtubule associated protein 2 (MAP-2), characteristic of neurons; glial fibrillary acidic protein (GFAP), present in astrocytes; galactocerebroside (GaIC) or myelin basic protein (MBP); characteristic of oligodendrocytes; Nestin, characteristic of neural precursors and other cells; and both A2B5 and NCAM, which appear on populations of differentiated from pPS cells capable of forming both neuronal cells and glial cells.

Hepatocyte lineage cells will typically display at least three of the following markers: α1-antitrypsin (AAT) synthesis, albumin synthesis, asialoglycoprotein receptor (ASGR) expression, absence of α-fetoprotein, evidence of glycogen storage, evidence of cytochrome p450 activity, and evidence of glucose-6-phosphatase activity. Hepatocyte precursors express α-fetoprotein, whereas mature hepatocytes may not.

For reprogramming that takes cells from one germ layer and makes them capable of forming cells of a different germ layer, the following markers can be used: α-fetoprotein or albumin synthesis (for endoderm), muscle-specific actin (for mesoderm), and MAP-2 (for ectoderm).

Tissue markers can be detected by a number of different techniques. Immunological techniques include immunocytochemistry for cell-surface markers, immunohistochemistry of fixed cells or tissue sections for intracellular or cell-surface markers, Western blot analysis of cellular extracts, and enzyme-linked immunoassay, for cellular extracts or products secreted into the medium. The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods, based on known sequence data.

To assay markers on reprogrammed cells cocultured with pPS cells, embryoid body cells, or instructor cells, it is useful to have a second marker that distinguishes the reprogrammed cells from other cells in the population. In many instances, the reprogramming or instructor cells can be selected to have a distinguishing inherent marker. For example, they can be selected to be of a different sex genetically, in which case they can be distinguished by fluorescence in situ hybridization using commercially available reagents for X or Y chromosomal markers. They can be selected to be of a different species, and distinguished by karyotype.

As another option, either the reprogramming or instructor cells, or the cells being reprogrammed can be labeled before the two populations are pooled in the coculture. One labeling method is to transfect the cells to express a marker gene, such as green fluorescent protein (pEGFP-C1, ClonTech cat. #6084-1), luciferase, lacZ, β-galactosidase, or an antibody-recognizable surface protein. Another labeling method is to label the cells with a transient marker that is detectable in the cell and its progeny after a few cell divisions, but becomes diluted out upon further proliferation. Such labels include radioisotopes (such as ¹²⁵I, ¹¹¹In, or [³⁵S]amino acids), fluorescent labels (such as fluorescein or Texas Red™), substrate homologs (such as bromodeoxyuridine), and various probes or stains (such as mitochondria labels dil and diO, and Hoechst Dyes). Those skilled in the art will recognized that detection of some labels (such as lacZ and β-galactosidase) involves a process incompatible with cell viability, and are better suited for use in protocol development and assay procedures.

In optimizing reprogramming protocols, co-localization of the label placed in the cells before reprogramming, and a tissue-specific marker for a different cell lineage correlates with successful reprogramming of the cell. Such labeling methods can also be used to keep track of reprogrammed cells placed in an animal to determine survival or subsequent further differentiation of the cells at a particular tissue site in vivo.

Telomerizing Reprogrammed Cells

Where it is desirable to increase the replicative capacity of the reprogrammed cells, they can be telomerized by genetically altering them to express telomerase reverse transcriptase. The catalytic component of human telomerase (hTERT) is provided in International Patent Publication WO 98/14592. For some applications, other TERT sequences can be used (mouse TERT is provided in WO 99/27113).

Typically, the vector will comprise a TERT encoding region under control of a heterologous promoter that will promote transcription in the reprogrammed cell. Optionally, a marker gene can be included in the same cassette to facilitate selection. Transfection and expression of telomerase in human cells is described in Bodnar et al., Science 279:349, 1998, and Jiang et al., Nat. Genet. 21:111, 1999. A method of telomerizing without genetically altering the cells is to pulse the cells with a TERT transcript or TERT protein. This may increase telomerase activity in the treated parental cell, but not after extensive proliferation.

Before and after telomerization, telomerase activity can be determined using TRAP activity assay (Kim et al., Science 266:2011, 1997), using a commercially available kit (TRAPeze® XK Telomerase Detection Kit, Cat. s7707; Intergen Co., Purchase N.Y.; or TeloTAGGG Telomerase PCR ELISAplus, Cat. 2,013,89; Roche Diagnostics, Indianapolis). hTERT expression can also be evaluated at the mRNA level by RT-PCR. The LightCycler TeloTAGGG hTERT quantification kit (Cat. 3,012,344; Roche Diagnostics) is available commercially for research purposes.

Telomerization of the cells can take place before or after reprogramming, and is particularly desirable if extensive proliferation of the cell is desired, either in vitro or after administration to a subject.

Other methods of immortalizing cells are also contemplated, such as genetically altering the cells with DNA encoding the SV40 large T antigen (U.S. Pat. No. 5,869,243, International Patent Publication WO 97/32972), infecting with EBV, or introducing oncogenes such as myc and ras. Transfection with oncogenes or oncovirus products is usually less suitable when the cells are to be used for human therapy.

Use of Reprogrammed Cells for Regenerative Therapy and Other Purposes

When the objective is to treat a patient with reprogrammed autologous cells, the original cell source will usually be a tissue that can be obtained by minimally invasive procedures. Non-limiting examples include the following:

Blood cells (such as CD34+ cells), obtainable by phlebotomy or leukapheresis

Bone marrow cells (which comprise both CD34+ hematopoietic cells and mesenchymal cells), obtainable by bilateral posterior iliac crest percutaneous bone marrow aspiration

Skin cells (which comprise dermal cells, fibroblasts, keratinocytes, and other cells)

Primary liver cell preparations (comprising proliferative hepatocytes and liver cell precursors) are obtainable by careful digestion of biopsy or surgically excised samples

Cells in a matched, preserved umbilical cord (which comprise both fibroblasts and cord blood cells, relatively rich in progenitors)

If desired, different cell types can be separated from mixed populations according to standard procedures illustrated in the references cited earlier. In some instances, separation of cell types may not be necessary.

Primary cell preparations will generally be stabilized in culture using known culture conditions suitable for the respective cell type, before they are reprogrammed. The reprogramming then takes place according to the principles of this invention, and prepared in a form suitable for human readministration. Typically, the preparation is tested to ensure that it is sterile, and can also be tested for the presence of phenotypic markers of the desired cell type. General procedures for the preparation of pharmaceutical preparations are provided in the most current edition of Remington's Pharmaceutical Sciences, or in Drug Products for Clinical Trials: An International Guide to Formulation, Production, and Quality Control (Monkhouse & Rhodes, eds., Marcel Dekker, 1998). Methods of cell therapy are generally described in Cell Therapy: Stem Cell Transplantation, Gene Therapy, and Cellular Immunotherapy (G. Morstyn, ed., 1996); and Cell Therapy (Y. Ikeda, Ed., Springer Verlag, 2000)

The pharmaceutical composition comprises the cells suspended in a suitable excipient, such as an isotonic buffer for injection intravenously or into a solid tissue mass, or a gel for administration into a cavity during a surgical procedure. The dose will depend on a number of factors, including the cell type, the degree of differentiation and proliferation capacity of the reprogrammed cell, and the extent of disease. As a general guide, it is estimated that the number of cells should be sufficient to reestablish the desired functional activity in one or more doses after about 3 to 10 population doublings, if the cells are expected to reach terminal differentiation soon after administration, or up to 20 doses or more if progenitor cells in the population have substantial self-renewing capacity. Ultimate determination of the administration protocol and the appropriate dose is under control of the managing clinician, and tailored to the patient being treated.

Monitoring the efficacy of treatment can comprise histopathology and immunocytology of blood samples (in the case of hematopoietic reconstitution) or biopsy samples taken near the site of treatment of a solid tissue. The ultimate goal is usually restoration of clinical function, determined according to the usual clinical markers for the disease being treated.

The techniques of this invention are also useful for treatment of a human patient with allogeneic cells obtained from a third-party donor, or from a tissue bank. This can be undertaken in a variety of circumstances, and is particularly appropriate when the cell type needed for therapy is difficult to obtain from healthy donors, and the patient is unable to supply adequate cells of their own for reprogramming. The procedure for collection, reprogramming, and administration of the cells is similar to that for autologous treatment, with the understanding that histocompatibility matching may be beneficial, and immunotolerization or immunosuppression of the patient may be necessary to avoid transplant rejection or graft-versus-host disease.

The techniques of this invention can also be used for any other worthwhile purpose. A further non-limiting example is reprogramming of cells for use in drug screening. For example, leukocytes produced as a byproduct of whole blood donation may be reprogrammed according to this invention into another cell type, useful for screening toxicity of a compound, or the ability of the test compound to modulate metabolism or differentiation of the cell. General principles for drug screening are provided in standard reference texts such as In vitro Methods in Pharmaceutical Research (Academic Press, 1997). A screening assay typically involves combining the compound with the reprogrammed cell, determining any phenotypic or metabolic changes in the progeny that result from contact with the compound, and correlating the change with toxicity or modulation potential of the compound.

The examples that follow are provided by way of further illustration, and are not meant to limit the claimed invention.

### EXAMPLES

### Example 1

Reprogramming for Neuronal Differentiation

Human mesenchymal stem cells (hMSC), genotype XY (Poietics, Cat. #PT-2501) were co-cultured with human embryonic stem (hES) cells, genotype XX, in a neuronal differentiation regime. The hMSCs in the population were then analyzed for the expression of neuronal markers.

The hMSC cells were maintained in MSC growth medium (Poietics, Cat.#PT-3001) and used before passage 5 for all experiments. The hES cells were maintained in feeder-free conditions as described in further detail elsewhere (WO 01/51616).

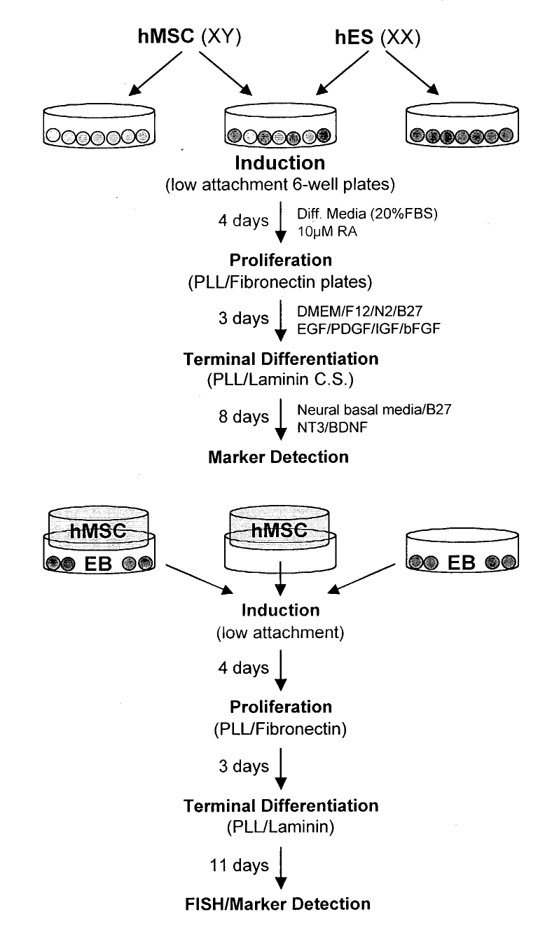

FIG. 1 shows the scheme for causing the hMSC to be reprogrammed. Cells were co-cultured using transwells in which the cells shared the same medium, or the cultures were set up as mixed hES/hMSC cultures allowing matrix and cell-cell interaction.

Confluent hES cells (or hMSC for cell-cell mixed co-cultures) were harvested by collagenase IV treatment, and cultured together as embryoid bodies (EBs) in suspension in induction medium containing 80% KO DMEM, 20% FBS (not heat-inactivated), 1% non-essential amino acid, 1 mM L-glutamine, 0.1 mM β-mercaptoethanol and 10 μM all-trans retinoic acid (RA) (Sigma). After 4 days, EBs were plated onto polylysine (PLL)/fibronectin coated plates or chamber slides and cultured for 3 days in DMEM/F-12 medium (BioWhittaker) containing N2-Supplement (Gibco), B27-Supplement (Gibco), 10 ng/mL human EGF (R&D System), 1 ng/mL human PDGF-AA (R&D System), 10 ng/mL hbFGF (Gibco) and 1 ng/mL human IGF-I (R&D System). Cells were then dissociated with trypsin/EDTA and seeded at 2.5×10⁴ cells cm⁻² into PLL/laminin-coated chamber slides. The cells were cultured for 1-2 weeks in Neurobasal Medium (Gibco) containing 2% B27 (Gibco) and supplemented with 10 ng/mL hNT-3 (Neurotrophin 3) (R&D System) and 10 ng/mL human BDNF (brain-derived neurotrophic factor) right before use.

In the transwell experiments, hMSC seeded onto the insert (5-6×10³ cells cm⁻², overnight) were co-cultured with hES-derived EBs for 4 days and plated on fibronectin-coated plates for 3 days using similar procedure as described above. Both hMSC and hES-derived cells were then dissociated and hES-derived cells were seeded onto the inserts and hMSC cells were seeded into chamber slides.

After differentiation, cultures were evaluated for neuronal formation using neuron-specific markers. hMSC were identified by fluorescence in-situ hybridization (FISH) analysis for the Y chromosome.

Immunocytochemistry and FISH analysis was conducted as follows. The cells were fixed with the solution containing 60% methanol, 10% acetic acid and 30% chloroform for 10 min at room temperature. The cells were then rinsed with PBS for 2-3 times, blocked with 5% normal goat serum (NGS) in PBS for 2 hrs and incubated with primary antibodies diluted in 1% NGS at RT for 1 hr. Cells were then washed with PBS and incubated with corresponding secondary antibodies.

After washing with PBS, cells were stained with DAPI or processed for Y chromosome detection by FISH using CEP chromosome enumeration DNA FISH probes (Vysis Inc.) according to manufacture's suggested procedures. Cells were counterstained with DAPI (Boehringer Cat.#236-276) (1:1000) at room temperature for 10-15 min and mounted with VectaShield™ mounting medium (Vector Cat.#H-1000).

For PS-NCAM staining, live cells were washed with PBS twice, incubated 1:1 diluted antibody 5A5, and incubated at 37° C. for 1 h. After washing in PBS twice, the cells were fixed as described above and washed in PBS twice. Cells were then incubated with Texas Red™ goat anti-mouse IgM (1:100 diluted in 1%NGS) at room temperature for 30 min. After washing in PBS for three times, cells were processed for FISH analysis and mounted after stained with DAPI.

Primary antibodies used were as follows. β-tubulin III: Sigma Cat. #T8860, diluted 1:1000. GFAP: DAKO Cat. #Z0334, diluted 1:500. MAP2: Sigma Cat. #M1406, diluted 1:500. Neurofilament: SternbergerCat. #SM1311, diluted 1:1000. 5A5: Developmental Hybridoma Bank, diluted 1:1. Results are shown in Tables 1 & 2.

TABLE 1
Marker Analysis of Cells Cultured in Transwells

| | | Day 8 | | Day 15 | | | |
|---|---|---|---|---|---|---|---|
| | | Neuro- | | β-tubulin III | | MAP2 | |
| Cells | Medium | filament | β-tubulin III | XX | XY | XX | XY |
| hES cells | neuronal medium | | | + | | + | |
| hMSC + hES cells | neuronal medium | + | + | + | − | + | − |
| hMSC | neuronal medium | + | + | | ± | | − |
| hMSC | MSC basal medium | + | + | | ± | | − |

TABLE 2
Marker Analysis of Cells Cocultured in Direct Contact

| | | Day 7 | | Day 18 | | | |
|---|---|---|---|---|---|---|---|
| | | PS-NCAM | | β-tubulin III | | MAP2 | |
| Cells | Medium | XX | XY | XX | XY | XX | XY |
| hES cells | neuronal medium | + | | + | | + | |
| hMSC + | neuronal medium | + | + | ++ | − | ++ | − |
| hES cells | | | | | | | |
| hMSC | neuronal medium | | + | | − | | ± |

At differentiation day 8, immunoreactivity of neurofilament and β-tubulin III were detected in hMSC and hES-derived cells. Weak expression of β-tubulin III was also found in hMSC at differentiation day 15, however, these cells lacked neuronal morphology.

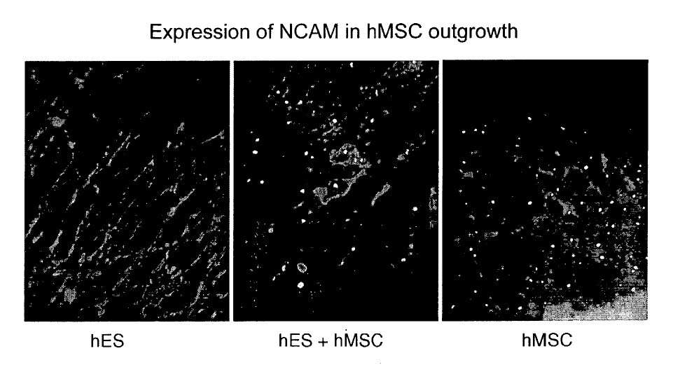

FIG. 2 shows cell markers observed in cell-cell contact cocultures. Confluent hES cells or hMSC were harvested by collagenase IV treatment and cultured together as embryoid bodies (EBs) in suspension in induction medium, and then immunostained as before. Some of the hMSC displayed neuronal morphology and expressed PS-NCAM at differentiation day 7. PS-NCAM expression was also found in hMSC cultured alone in neuronal differentiation medium. After cells were dissociated and cultured on laminin for 11 days (at differentiation day 18), weak β-tubulin III but no MAP2 expression was found in hMSC (Table 1).

### Example 2

Reprogramming for Hepatocyte Differentiation

To demonstrate reprogramming and hepatocyte differentiation potential of hMSC, hMSC (XY) were cocultured with hES (XX) cells, and then subject to hepatocyte differentiation conditions.

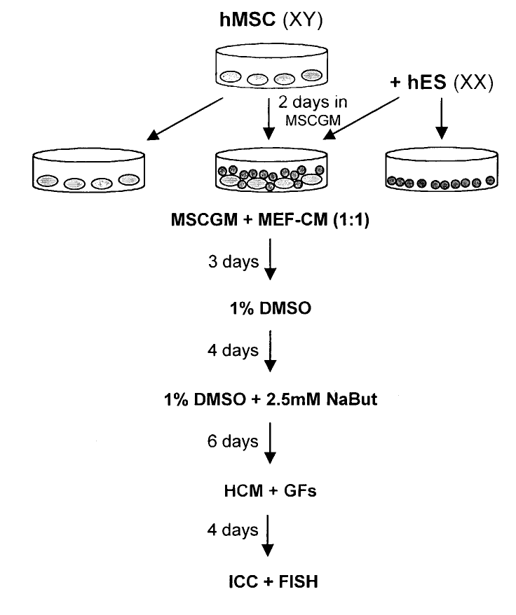

FIG. 3 shows the scheme used in this experiment. HMSC cells (XY) were seeded onto chamber slides at 6×10³ cells cm⁻² and maintained in MSCGM for 2 days before coculturing with hES cells (XX). Confluent hES cells were harvested by treatment with 200 units/mL collagenase IV and seeded onto the hMSC culture for the coculture or on Matrigel-coated chamber slides as a control. HMSC culture alone was also used as a control. These 3 cultures were then maintained in 1:1 mixture of MSCGM and MEF-CM for 3 days before changing to KO-DMEM containing 20% serum replacement (Gibco BRL), 1% non-essential amino acids, 1 mM glutamine, 0.1 mM p-mercaptoethanol and 1% DMSO (ATCC).

Cells were differentiated into hepatocytes by supplementing the medium with 2.5 mM sodium butyrate (Sigma) for 6 days and finally placed into HCM (Clonetics) supplemented with 2.5 ng/mL HGF (Calbiochem), and 10 ng/mL TGFα Calbiochem) for 4 more days.

Immunocytochemistry and FISH analysis was conducted as described in Example 1. The primary antibody to albumin was obtained from Sigma, and diluted 1:200

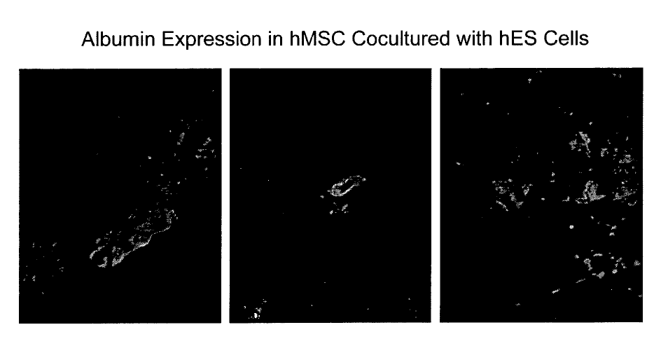

FIG. 4 shows the results. After 17 days of co-culture, cells were identified which were albumin positive and Y chromosome positive, showing they were derived from the hMSC cells in the population. In contrast, hMSC cultured alone did not show albumin immunoreactivity.

The data indicate that hMSC differentiate into hepatocyte-like cells when cocultured with hES cells (thought to cause reprogramming) and then subject to a hepatocyte differentiation paradigm.

The compositions and procedures provided in the description can be effectively modified by those skilled in the art without departing from the spirit of the invention embodied in the claims that follow.

The invention claimed is:

1. A method of reprogramming a human cell, comprising culturing the cell in the presence of primate pluripotent stem (pPS) cells, embryoid body cells, or a cell lysate or conditioned medium prepared from such cells.

2. The method of claim 1, wherein the human cell is a restricted lineage precursor cell.

3. The method of claim 2, wherein the restricted lineage precursor cell is selected from CD34+ leukocytes, cord blood cells, mesenchymal stem cells, stromal cells, neural stem cells, and primary liver cells.

4. The method of claim 1, wherein the cell or its progeny is cultured with or passaged into a growth environment that comprises an extracellular matrix and a medium supplemented with components that promote differentiation of the cell.

5. The method of claim 1, wherein the cell or its progeny is cultured with or passaged into a growth environment that comprises cells of a lineage different from the lineage of the cell being reprogrammed.

6. The method of claim 4, wherein the growth environment promotes differentiation of the cell or its progeny to cells of the neural or hepatocyte lineage.

7. The method of claim 1, wherein the pPS cells are human embryonic stem (hES) cells.

8. The method of claim 1, comprising culturing the cell in the presence of pPS cells.

9. The method of claim 1, comprising culturing the cell in the presence of embryoid body cells.

10. The method of claim 1, comprising culturing the cell in the presence of cell lysate prepared from pPS cells.

11. The method of claim 1, comprising culturing the cell in the presence of cell lysate prepared from embryoid body cells.

12. The method of claim 1, comprising culturing the cell in the presence of medium conditioned by pPS cells.

13. The method of claim 1, comprising culturing the cell in the presence of medium conditioned by embryoid body cells.

14. The method of claim 1, further comprising telomerizing the cell before or after the reprogramming.

15. A reprogrammed human cell, prepared according to the method of claim 1.

16. A kit for reprogramming a human cell according to claim 1, comprising pPS cells or embryoid body cells; and written instructions for the use of the cells for reprogramming.

17. A kit for reprogramming a human cell according to claim 1, comprising a cell lysate prepared from pPS cells or embryoid body cells; and written instructions for the use of the lysate for reprogramming.

18. A kit for reprogramming a human cell according to claim 1, comprising a medium conditioned by pPS cells or embryoid body cells; and written instructions for the use of the medium for reprogramming.

19. A composition for reprogramming a human cell according to claim 1, comprising:
a) the cell to be reprogrammed, selected from CD34+ leukocytes, cord blood cells, mesenchymal stem cells, stromal cells, neural stem cells, and primary liver cells; in combination with
b) primate pluripotent stem (pPS) cells or embryoid body cells having a different genotype from the cell being reprogrammed, or a cell lysate or conditioned medium prepared from such cells.

20. A method for preparing a pharmaceutical composition, comprising reprogramming a population of human cells according to claim 1, and combining the reprogrammed cell with a pharmaceutical excipient.

21. A method for evaluating the effectiveness of a cell population for reprogramming a cell in a coculture system, comprising coculturing the cell to be reprogrammed with the cell population, simultaneously or sequentially culturing the cell in a growth environment that promotes differentiation of the cell or its progeny into a new lineage that is different from the lineage of the cell before coculturing with the cell population, and evaluating the effectiveness of the cell population based on whether the ability of the cell to produce progeny of the new lineage has been increased.

22. A method for evaluating the effectiveness of a medium for reprogramming a cell according to claim 1, comprising culturing the cell in the medium, simultaneously or sequentially culturing the cell in a growth environment that promotes differentiation of the cell or its progeny into a new lineage that is different from the lineage of the cell before culturing in the medium, and evaluating the effectiveness of the medium based on whether the ability of the cell to produce progeny of the new lineage has been increased.

23. A method for evaluating the effectiveness of a cell lysate for reprogramming a cell according to claim 1, comprising culturing the cell in the presence of the lysate, simultaneously or sequentially culturing the cell in a growth environment that promotes differentiation of the cell or its progeny into a new lineage that is different from the lineage of the cell before culturing with the lysate, and evaluating the effectiveness of the lysate based on whether the ability of the cell to produce progeny of the new lineage has been increased.

24. A method for screening a compound for toxicity or modulation potential for a particular cell type, comprising combining the compound with progeny of the particular cell type grown from a cell reprogrammed according to claim 1, determining any phenotypic or metabolic changes in the progeny that result from contact with the compound, and correlating the change with the toxicity or modulation potential.

25. A method for treating a patient to supplement activity of a particular cell type, comprising obtaining from the patient cells of a different lineage from that of the cell type, reprogramming the cells according to claim 1 so that the reprogrammed cells can produce progeny of the particular cell type, and then readministering the reprogrammed cells to the patient.
